(19) United States
(12) Patent Application Publication
(10) Pub. No.: US 2005/0238603 A1
(43) Pub. Date: Oct. 27, 2005

(54) Anhydrous cosmetic composition comprising at least one oil, at least one polymeric gelling agent and at least one polyurethane powder

(76) Inventors: Agnes Themens, Bourg La Reine (FR); Maitena Leuridan, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/099,528

(22) Filed: Apr. 6, 2005

Related U.S. Application Data
(60) Provisional application No. 60/569,648, filed on May 11, 2004.

(30) Foreign Application Priority Data
Apr. 6, 2004 (FR) 04 50696

(51) Int. Cl. A61K 7/06; A61K 7/11

(52) U.S. Cl. 424/70.11

(57) ABSTRACT

An anhydrous composition for making up and/or caring for the skin comprising an oily phase, at least one amorphous gelling polymer formed by polymerization of at least one olefin and at least one polyurethane powder. This composition exhibits a smooth and melting cream texture, spreads easily over the skin and may make it possible to obtain a homogeneous make-up which has a powdery finish and which confers a pleasant softness on the skin; the composition also exhibits good hold properties.

This application claims benefit of U.S. Provisional Application No. 60/569,648, filed May 11, 2004, the contents of which are incorporated herein by reference. This application also claims benefit of priority under 35 U.S.C. § 119 to French Patent Application No. FR 04/50696 filed Apr. 6, 2004, the contents of which are also incorporated by reference.

Disclosed herein is an anhydrous cosmetic composition for making up and/or caring for the skin. Further disclosed herein is a process for making up and/or caring for human skin comprising applying such a composition to the skin.

The composition for making up the skin can be in a form chosen from a foundation, an eye shadow, a blusher, a concealer, and a product for making up the body. In one embodiment, the composition is a foundation composition.

The care composition can, for example, be in a form chosen from a make-up base, a mattifying product for the skin, and a product for caring for the skin.

In one embodiment, the composition may be a composition for making up the skin.

Foundation compositions are commonly employed to contribute an attractive color to the skin, for example, the face, but also to conceal the imperfections of the skin, such as red blotches or blemishes.

These compositions may have varied textures, ranging from fluid to solid, and generally comprise oils and pulverulent coloring materials. At least one of the difficulties encountered by users is that of being able to uniformly spread the foundation over the entire surface of the face, so as to uniformly distribute the product. Compositions with thick or solid textures may be difficult to spread because of their high viscosities. Compositions with fluid textures may not always be appropriate for producing a uniform make-up, for example, not leaving visible marks on the skin because of their poor spreading over the entire surface of the face to be made up. Furthermore, the presence of pulverulent materials can produce a desiccating effect on the make-up, resulting in a feeling of tightness, thus rendering the make-up uncomfortable to wear throughout the day.

Consumers are always on the lookout for products with innovative texture effects. For example, products are sought which exhibit different textures before and after application of the product for making up and/or caring for the skin.

It would be desirable, therefore, to have available a make-up and/or care composition which has the appearance of a smooth cream and which is converted to a powdery finish after its application to the skin. It would also be desirable to have available a composition that exhibits a feeling of softness when it is applied to the skin, and/or which exhibits at least one good hold property, for example, hold with regard to sebum or sweat, for example, after twelve hours following application of the composition.

The present inventors have discovered that such a composition is obtained by using in combination at least one oil, at least one specific polymeric gelling agent, and at least one polyurethane powder.

More specifically, disclosed herein is an anhydrous composition for making up and/or caring for skin comprising an oily phase, at least one amorphous gelling polymer formed by polymerization of at least one olefin, and at least one polyurethane powder.

Even further disclosed herein is a cosmetic process for making up and/or for the non-therapeutic treatment of skin, comprising applying to the skin at least one anhydrous composition as defined above.

The composition disclosed herein may exhibit a smooth creamy texture that melts when it is applied to the skin. The composition may be spread easily over the skin and may make it possible to obtain a uniform make-up of the skin without leaving visible traces. Furthermore, after application of the composition disclosed herein to the skin, the make-up or the deposited layer obtained may exhibit a powdery and velvety finish and may be comfortable to wear, without a desiccating or tightness effect; the made-up or treated skin may exhibit a pleasant softness. Furthermore, the make-up obtained may have good hold properties, for example, good hold with respect to sebum or to sweat: after a time period of 12 hours after application of the composition, the make-up may still remain on the skin and may be homogeneous.

Further disclosed herein is the use of the composition as defined above to produce a make-up having at least one of the following properties: good hold, for example, after 12 hours, a soft made-up skin, a comfortable make-up, a make-up having a powdery appearance, and/or a homogeneous make-up.

As used herein the term “anhydrous composition” means a composition comprising no more than 2% by weight of water, such as no more than 0.5% of water, and which may be, for example, devoid of water, wherein the water is not added during the preparation of the composition but corresponding to the residual water introduced when the ingredients are mixed.

The at least one polymeric gelling agent present in the composition disclosed herein may be chosen from amorphous polymers formed by polymerization of at least one olefin. As used herein, the term “amorphous polymer” means a polymer that does not have a crystalline form. The at least one olefin may, for example, be chosen from elastomeric monomers having ethylenic unsaturation.

Mention may be made, as examples of the at least one olefin, of ethylenic carbon monomers having, for example, one or two ethylenic unsaturations and comprising from 2 to 5 carbon atoms, such as ethylene, propylene, butadiene, and isoprene.

The at least one polymeric gelling agent is capable of thickening or gelling the organic phase of the composition. The at least one polymeric gelling agent is also film-forming, that is to say that it is capable of forming a film when it is applied to the skin.

The at least one polymeric gelling agent may, for example, be chosen from diblock, triblock, multiblock, radial, and star copolymers. In one embodiment, the at least one polymeric gelling agent is chosen from triblock, multiblock, radial, and star copolymers.

Such polymeric gelling agents are disclosed in U.S. Patent Application No. U.S. 2002/005562 and in U.S. Pat. No. 5,221,534.

In one embodiment, the at least one polymeric gelling agent is chosen from amorphous block copolymers of styrene and of olefin.

In one embodiment, the at least one polymeric gelling agent may be hydrogenated in order to reduce the residual ethylenic unsaturations after the polymerization of the monomers. For example, the at least one polymeric gelling agent may be an optionally hydrogenated copolymer comprising styrene blocks and comprising ethylene/C₃-C₄ alkylene blocks.

Mention may be made, as examples of the hydrogenated diblock copolymer, of styrene-ethylene/propylene copolymers and styrene-ethylene/butadiene copolymers. Diblock polymers are sold, for example, under the name Kraton® G1701E by Kraton Polymers.

Mention may be made, as examples of the hydrogenated triblock copolymer, of styrene-ethylene/propylene-styrene copolymers, styrene-ethylene/butadiene-styrene copolymers, styrene-isoprene-styrene copolymers, and styrene-butadiene-styrene copolymers. Triblock polymers are sold, for example, under the names Kraton® G1650, Kraton® G1652, Kraton® D1101, Kraton® D1102, and Kraton® D1160 by Kraton Polymers.

Use may also be made of a blend of hydrogenated styrene-butylene/ethylene-styrene triblock copolymers and hydrogenated ethylene-propylene-styrene star polymers, such a blend being, for example, in isododecane. Such blends are, for example, sold by Penreco under the trade names Versagel® M5960 and Versagel® M5670.

In one embodiment, the at least one polymeric gelling agent is a diblock copolymer such as those described above, for example, a styrene-ethylene/propylene diblock copolymer.

The at least one polymeric gelling agent may be present in an amount ranging from 0.1% to 10% by weight, relative to the total weight of the composition, for example, ranging from 0.5% to 5% by weight, and, further, for example, ranging from 1% to 3% by weight.

As noted above, the composition disclosed herein comprises at least one polyurethane powder. In at least one embodiment, the at least one polyurethane powder is not film-forming, that is to say that it does not form a continuous film when it is deposited on a substrate such as the skin.

For example, the at least one polyurethane powder may be a powder formed of hexamethylene diisocyanate and trimethylol hexyllactone copolymer. Such a polyurethane powder is sold, for example, under the names “Plastic Powder D-400” and “Plastic Powder D-800” by Toshiki.

Use may be made, as another example of the at least one polyurethane powder, of powders sold under the name “Plastic Powder CS400” by Toshiki.

The at least one polyurethane powder can be present in an amount ranging from 0.5% to 30% by weight, relative to the total weight of the composition, and, for example, ranging from 1% to 15% by weight, and, further, for example, ranging from 5% to 15% by weight.

According to one embodiment, the composition can comprise poly(methyl methacrylate) particles. More specifically, the poly(methyl methacrylate) particles are not film-forming, i.e., they do not form a continuous film when they are deposited on a substrate such as the skin.

The powders formed of poly(methyl methacrylate) are generally provided in the form of white-colored hollow or solid spherical particles, the number-average size of which is generally on the scale of a micrometer and, for example, ranges from 3 to 15 microns such as from 3 to 10 microns. The term “number-average size” means the dimension given by the random particle size distribution at half the population, referred to as D50.

It is also possible to characterize these poly(methyl methacrylate) particles by their density, the latter being capable of ranging, for example, according to the size of the spherical cavity of the particles.

In the context of the present disclosure, this density is assessed according to the following protocol, referred to as packed density:

Powder, w=40 g, is poured into a graduated measuring cylinder; then the measuring cylinder is placed on the STAV 2003 device from Stampf Volumeter; the measuring cylinder is subsequently subjected to 1500 packing motions; then the final volume Vf of packed powder is measured directly on the measuring cylinder. The packed density is determined by the ratio w/Vf, as it happens 40/Vf (wherein Vf is expressed in cm³ and w in g).

For example, the density of the poly(methyl methacrylate) particles which can be used can range from 0.3 to 1.5, for example, from 0.5 to 1.5 and, even further, for example, from 1 to 1.5.

Examples of poly(methyl methacrylate) particles suitable for use in the composition disclosed herein include the poly(methyl methacrylate) particles sold by Matsumoto Yushi Co. under the name “Micropearl M100”, by LCW under the name “Covabead LH85” and by Nihon Junyaku under the name “Jurymer MB1”.

The poly(methyl methacrylate) particles can be present in an amount ranging from 0.5% to 30% by weight, relative to the total weight of the composition, for example, ranging from 0.5% to 25% by weight, further, for example, ranging from 1% to 20% by weight, and, even further, for example, ranging from 5% to 15% by weight.

The poly(methyl methacrylate) powder may make it possible, for example, to obtain a make-up or mattifying care product.

The oily phase of the composition disclosed herein comprises at least one oil which can be chosen from volatile oils and non-volatile oils. In one embodiment, the composition comprises at least one volatile oil and at least one non-volatile oil.

In one embodiment, the composition disclosed herein comprises at least one volatile oil.

As used herein, the term “volatile oil” means any oil capable of evaporating on contact with the skin at ambient temperature and atmospheric pressure. The at least one volatile oil may be chosen from volatile cosmetic oils which are liquid at ambient temperature and which have a non-zero vapour pressure, at ambient temperature and atmospheric pressure, ranging, for example, from 0.13 Pa to 40 000 Pa (0.001 to 300 mmHg) and, further, for example, ranging from 1.3 to 1300 Pa (0.01 to 10 mmHg).

The at least one volatile oil can be chosen from volatile hydrocarbon oils, volatile silicone oils, and volatile fluorinated oils.

As used herein, the term “hydrocarbon oil” is understood to mean an oil comprising mainly hydrogen and carbon atoms and optionally oxygen, nitrogen, sulphur and/or phosphorus atoms.

The volatile hydrocarbon oils can be chosen from hydrocarbon oils comprising from 8 to 16 carbon atoms, and, for example, branched C₈-C₁₆ alkanes, such as C₈-C₁₆ isoalkanes of petroleum origin (also referred to as isoparaffins), such as isododecane (also referred to as 2,2,4,4,6-pentamethylheptane), isodecane, isohexadecane and, for example, the oils sold under the trade names of Isopars® and Permethyls®.

The at least one volatile oil may also be chosen from volatile silicones, for example, volatile linear and cyclic silicone oils, such as those having a viscosity ≦5 centistokes (5×10⁻⁶ m²/s) and comprising, for example, from 2 to 10 silicon atoms, such as from 2 to 7 silicon atoms, these silicones optionally comprising at least one group chosen from alkyl and alkoxy groups comprising from 1 to 10 carbon atoms. Examples of volatile silicone oils include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

The volatile fluorinated oil generally does not have a flash point.

Examples of volatile fluorinated oils include nonafluoroethoxybutane, nonafluoromethoxybutane, decafluoropentane, tetradecafluorohexane, dodecafluoropentane and mixtures thereof.

For example, the composition comprises at least one volatile hydrocarbon oil, such as a mixture of isododecane and isohexadecane.

The at least one volatile oil can be present in the composition disclosed herein in an amount ranging from 5% to 60% by weight, relative to the total weight of the composition, for example, ranging from 10% to 55% by weight, further, for example, ranging from 20% to 50% by weight and, even further, for example, ranging from 30% to 50% by weight.

In one embodiment, the composition disclosed herein can comprise at least one non-volatile oil.

As used herein, the term “non-volatile oil” is understood to mean an oil which remains on the skin at ambient temperature and atmospheric pressure for at least several hours and which has, for example, a vapor pressure of less than 0.13 Pa (0.01 mmHg).

The at least one non-volatile oil can be chosen from hydrocarbon oils, for example, of animal or vegetable origin, and silicone oils. As used herein, the term “hydrocarbon oil” is understood to mean an oil comprising mainly hydrogen and carbon atoms and optionally oxygen, nitrogen, sulphur and/or phosphorus atoms.

The at least one non-volatile oil can, for example, be chosen from non-volatile hydrocarbon oils, if appropriate fluorinated, and non-volatile silicone oils.

Examples of the at least one non-volatile hydrocarbon oil include:
hydrocarbon oils of animal origin;
hydrocarbon oils of vegetable origin, such as triglycerides comprising esters of fatty acids and glycerol, the fatty acids of which can have various chain lengths from C₄ to C₂₄, it being possible for these chains to be linear or branched and saturated or unsaturated; these oils may be chosen from triglycerides of heptanoic acid and of octanoic acid; wheat germ, sunflower, grape seed, sesame, corn, apricot, castor, karite, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower, and musk rose oil; karite butter; and triglycerides of caprylic/capric acids, such as those sold by Stéarineries Dubois and those sold under the names Miglyol 810®, 812® and 818® by Dynamit Nobel;
synthetic ethers comprising from 10 to 40 carbon atoms;
linear and branched hydrocarbons of mineral and synthetic origin, such as liquid petrolatum, polydecenes, hydrogenated polyisobutene, such as Parleam®, squalane, and liquid paraffins;
synthetic esters, such as oils of formula R₁COOR₂, wherein R₁ is a residue of a linear or branched fatty acid comprising from 1 to 40 carbon atoms and R₂ is chosen from hydrocarbon chains, for example, branched hydrocarbon chains, comprising from 1 to 40 carbon atoms, provided that R₁+R₂ is ≧10, such as Purcellin oil (cetearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, and heptanoates, octanoates, decanoates and ricinoleates of alcohols and of polyalcohols, such as propylene glycol dioctanoate; hydroxylated esters, such as isostearyl lactate, diisostearyl malate and 2-octyldodecyl lactate; polyol esters and pentaerythritol esters;
fatty alcohols which are liquid at ambient temperature with a branched and/or unsaturated carbon chain comprising from 12 to 26 carbon atoms, such as octyidodecanol, isostearyl alcohol, oleyl alcohol, 2-hexyldecanol, 2-butyloctanol and 2-undecylpentadecanol; and
higher fatty acids, such as oleic acid, linoleic acid and linolenic acid.

The non-volatile silicone oils which can be used in the composition disclosed herein can be chosen from non-volatile polydimethylsiloxanes (PDMSs); polydimethylsiloxanes comprising pendant alkyl or alkoxy groups and/or alkyl or alkoxy groups at the end of the silicone chain, wherein the groups each comprise from 2 to 24 carbon atoms, or phenylated silicones, such as phenyl trimethicones, phenyl dimethicones, phenyl(trimethylsiloxy)diphenylsiloxanes, diphenyl dimethicones, diphenyl(methyldiphenyl)trisiloxanes, and mixtures thereof.

In one embodiment, the at least one non-volatile oil can be chosen from C₁₂-C₃₆ esters, such as those described above.

The at least one non-volatile oil can be present in an amount ranging from 0.5% to 60% by weight, relative to the total weight of the composition, for example, ranging from 1% to 50% by weight, further, for example, ranging from 5% to 40% by weight, even further, for example, ranging from 5% to 30% by weight, and, even further, for example, ranging from 10% to 20% by weight.

For example, when the composition disclosed herein comprises poly(methyl methacrylate) particles, the contents of the at least one non-volatile oil and of the poly(methyl methacrylate) particles are adjusted so that the amount of the at least one non-volatile oil is greater than the amount of poly(methyl methacrylate) particles. For example, these amounts are such that the non-volatile oil/poly(methyl methacrylate) particles ratio by weight can be greater than or equal to 1:1, for example, ranging from 1:1 to 35:1, further, for example, ranging from 1:1 to 25:1 and, even further, for example, ranging from 1:1 to 15:1; for example, the non-volatile oil/poly(methyl methacrylate) particles ratio by weight can be greater than or equal to 1.5:1, for example, ranging from 1.5:1 to 35:1, further, for example, ranging from 1.5:1 to 25:1 and, even further, for example, ranging from 1.5:1 to 15:1.

The composition disclosed herein can further comprise at least one coloring material, for example, chosen from pigments, pearlescent agents, and fat-soluble dyes.

As used herein, the term “pigments” should be understood as meaning white or colored and inorganic or organic particles of any shape which are insoluble in the physiological medium and which are intended to color the composition.

As used herein, the term “pearlescent agents” should be understood as meaning iridescent particles of any shape produced, for example, by certain shellfish in their shells or else synthesized.

As used herein, the term “dyes” should be understood as meaning compounds, generally organic compounds, which are soluble in fatty substances, such as oils.

The pigments can be chosen from white and colored and inorganic and organic pigments. Examples of inorganic pigments include titanium dioxide, optionally surface-treated, zirconium and cerium oxides, and zinc, iron (black, yellow and red) and chromium oxides, manganese violet, ultramarine blue, chromium hydrate and ferric blue, and metal powders, such as aluminium powder and copper powder.

Examples of organic pigments include carbon black, D & C pigments, and lakes, based on cochineal carmine, of barium, strontium, calcium and aluminium.

The pearlescent pigments can be chosen from white pearlescent pigments, such as mica covered with titanium oxide or with bismuth oxychloride, colored pearlescent pigments, such as titanium oxide-coated mica covered with iron oxides, titanium oxide-coated mica covered with, for example, ferric blue or chromium oxide, and titanium oxide-coated mica covered with an organic pigment of the abovementioned type, and pearlescent pigments based on bismuth oxychloride.

The fat-soluble dyes may, for example, be chosen from Sudan red, D & C Red No. 17, D & C Green No. 6, β-carotene, soybean oil, Sudan brown, D & C Yellow No. 11, D & C Violet No. 2, D & C Orange No. 5, quinoline yellow, and natto and bromoacids.

The at least one coloring material may be present in an amount ranging from 0.1% to 30% by weight, relative to the total weight of the composition, for example, ranging from 0.1% to 20% by weight, for example, ranging from 0.5% to 15% by weight, further, for example, ranging from 1% to 15% by weight and, even further, for example, ranging from 5% to 15% by weight.

The composition disclosed herein can also comprise at least one additional filler other than the at least one polyurethane powder and the poly(methyl methacrylate) particles described above.

As used herein, the term “fillers” should be understood as meaning colorless or white and inorganic or synthetic particles of any shape which are insoluble in the medium of the composition, whatever the temperature at which the composition is manufactured.

The at least one additional filler can be inorganic or organic and of any shape—platelet, spherical or oblong, whatever the crystallographic form (for example sheet, cubic, hexagonal, orthorhombic, and the like). Mention may be made of talc, mica, silica, kaolin, polyamide (Nylon®) powders, poly-β-alanine powders, polyethylene powders, the powders formed of tetrafluoroethylene polymers (Teflon®), lauroyllysine, starch, boron nitride, polymeric hollow microspheres, such as those of poly(vinylidene chloride)/acrylonitrile, for example Expancel® (Nobel Industrie), and of acrylic acid copolymers, silicone resin powders, for example, silsesquioxane powders (silicone resin powders disclosed, for example, in Patent No. EP 293 795; Tospearls® from Toshiba, for example), polyorganosiloxane elastomer particles, precipitated calcium carbonate, magnesium carbonate, basic magnesium carbonate, hydroxyapatite, hollow silica microspheres, glass and ceramic microcapsules, metal soaps derived from organic carboxylic acids comprising from 8 to 22 carbon atoms, such as from 12 to 18 carbon atoms, for example, zinc stearate, magnesium stearate, lithium stearate, zinc laurate, and magnesium myristate.

The at least one additional filler may be present in the composition disclosed herein in an amount ranging from 0.1% to 35% by weight, relative to the total weight of the composition, for example, ranging from 0.5% to 30% by weight and, further, for example, ranging from 1% to 25% by weight.

In one embodiment, the at least one additional filler is chosen from silsesquioxane powders, talc, and polyamide (Nylon®) powders.

The composition disclosed herein can comprise the silsesquioxane powders in an amount ranging from 0.5% to 30% by weight, relative to the total weight of the composition, and, for example, ranging from 1% to 15% by weight, and, further, for example, ranging from 1% to 10% by weight.

The composition disclosed herein can comprise the talc in an amount ranging from 0.1% to 30% by weight, relative to the total weight of the composition, and, for example, ranging from 1% to 15% by weight and, further, for example, ranging from 1% to 10% by weight.

The composition disclosed herein can comprise the polyamide powder in an amount ranging from 0.5% to 30% by weight, relative to the total weight of the composition, and, for example, ranging from 1% to 15% by weight, and, further, for example, ranging from 1% to 10% by weight.

In one embodiment, the composition disclosed herein can comprise a total amount of pulverulent material ranging from 20% to 50% by weight, relative to the total weight of the composition, for example, ranging from 25% to 45% by weight and, further, for example, ranging from 30% to 40% by weight.

The composition can also comprise at least one inorganic thickener, in the oily phase, for example, chosen from organophilic clay and pyrogenic silicas.

The organophilic clays are chosen from clays modified by chemical compounds that render the clay capable of swelling in oily media.

The clays are products already well known per se which are described, for example, in the work “Minéralogie des argiles [Mineralogy of Clays], S. Caillère, S. Hénin, M. Rautureau, 2nd edition, 1982, Masson”, the teaching of which is incorporated herein by reference.

The clays are silicates comprising a cation that can be chosen from at least one of calcium, magnesium, aluminium, sodium, potassium, and lithium cations.

Examples of clays that can be used include smectites, such as montmorillonites, hectorites, bentonites, beidellites and saponites, and the family of vermiculites, stevensite and chlorites.

These clays can be of natural or synthetic origin. For example, use can be made of the clays that are cosmetically compatible and acceptable with keratinous substances, such as skin.

The organophilic clay can be chosen from at least one of montmorillonite, bentonite, hectorite, attapulgite, and sepiolite. In one embodiment, the clay is chosen from bentonite and hectorite.

These clays can be modified with a chemical compound chosen from at least one of quaternary ammoniums, tertiary amines, amine acetates, imidazolines, amine soaps, fatty sulphates, alkylarylsulphonates, and amine oxides.

Mention may be made, as organophilic clays, of quaternary-18 bentonites, such as those sold under the names Bentone 3, Bentone 38 and Bentone 38V by Rheox, Tixogel VP by United Catalyst and Claytone 34, Claytone 40 and Claytone XL by Southern Clay; stearalkonium bentonites, such as those sold under the names Bentone 27 by Rheox, Tixogel LG by United Catalyst and Claytone AF and Claytone APA by Southern Clay; and quaternium-18/benzalkonium bentonite, such as those sold under the names Claytone HT and Claytone PS by Southern Clay.

Pyrogenic silicas can be obtained by high temperature hydrolysis of a volatile silicon compound in an oxyhydrogen flame, producing a finely divided silica. This process makes it possible, for example, to obtain hydrophilic silicas that exhibit a large number of silanol groups at their surfaces. Such hydrophilic silicas are sold, for example, under the names “Aerosil 130®”, “Aerosil 200®”, “Aerosil 255®”, “Aerosil 300®” and “Aerosil 380®” by Degussa and “Cab-O-Sil HS-5®”, “Cab-O-Sil EH-5®”, “Cab-O-Sil LM-130®”, “Cab-O-Sil MS-55®” and “Cab-O-Sil M-5®” by Cabot.

It is possible to chemically modify the surface of the silica by a chemical reaction which generates a decrease in the number of silanol groups. It is possible, for example, to substitute silanol groups by hydrophobic groups: a hydrophobic silica is then obtained.

The hydrophobic groups can be chosen from the following:
trimethylsiloxyl groups, which are obtained, for example, by treatment of pyrogenic silica in the presence of hexamethyldisilazane. Silicas thus treated are named “Silica silylate” according to the CTFA (6th edition, 1995). They are sold, for example, under the references “Aerosil R812®” by Degussa and “Cab-O-Sil TS-530®” by Cabot.
dimethylsilyloxyl and polydimethylsiloxane groups, which are obtained, for example, by treatment of pyrogenic silica in the presence of polydimethylsiloxane or of dimethyldichlorosilane. Silicas thus treated are named “Silica dimethyl silylate” according to the CTFA (6th edition, 1995). They are sold, for example, under the references “Aerosil R972®” or “Aerosil R974®” by Degussa or “Cab-O-Sil TS-610®” or “Cab-O-Sil TS-720®” by Cabot.

The pyrogenic silica may, for example, exhibit a particle size which can be nanometric to micrometric, for example, ranging from 5 to 200 nm.

The at least one inorganic thickener, in the oily phase, can be present in the composition disclosed herein in an amount ranging from 0.5% to 5% by weight, relative to the total weight of the composition, further, for example, in an amount ranging from 1% to 4% by weight and, even further, for example, ranging from 2% to 4% by weight.

The composition disclosed herein can further comprise at least one additional cosmetic ingredient which can be chosen, for example, from antioxidants, fragrances, preservatives, neutralizing agents, surfactants, waxes, sunscreen agents, vitamins, moisturizing agents, self-tanning compounds, and antiwrinkle active principles.

Of course, a person skilled in the art will take care to choose this or these optional additional compounds and/or their amounts so that the advantageous properties of the composition disclosed herein are not, or not substantially, detrimentally affected by the envisaged addition.

In one embodiment, the composition disclosed herein is provided in the form of a deformable non-fluid gel: the composition does not flow under its own weight at 25° C. in less than 5 minutes and it can be deformed by simple crushing when the composition is taken up with the fingers (contrary to a solid composition, which does not deform when it is brought into simple contact with the fingers).

According to another embodiment, the composition disclosed herein is devoid of polymer particles dispersed in the oily phase of the composition and stabilized at the surface by a stabilizing polymer. Such stabilized polymer dispersions are disclosed, for example, in Patent Application No. EP-A-749 747. The term “devoid of” is understood to mean that the composition comprises less than 3% by weight, relative to the total weight of the composition, of polymer particles dispersed and stabilized at the surface with a stabilizing polymer, indeed even does not comprise such particles. For example, these stable dispersions have the distinctive feature of remaining in the dispersion form when they are diluted to 50% by volume with their dispersing medium.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The disclosed embodiments are illustrated in more detail by the non-limiting example described below.

### EXAMPLE 1

A foundation having the following composition was prepared:

| | | |
|---|---|---|
| Isononyl isononanoate | 15.7 | g |
| Isododecane | 11.75 | g |
| Isohexadecane | 14.1 | g |
| Sorbitan monoisostearate | 1.5 | g |
| Styrene-ethylene/propylene copolymer, | 2.5 | g |
| sold under the name Kraton G 1701 E by | | |
| Kraton Polymers | | |
| Quaternary-18 hectorite gel, sold under | 17.65 | g |
| the name “Bentone gel SS 71 V” by Elementis | | |
| Polyurethane powder, sold under the name | 8 | g |
| “Plastic Powder D-400” by Toshiki | | |
| Poly(methyl methacrylate) powder | 9 | g |
| Polymethylsilsesquioxane powder, sold | 1.25 | g |
| under the name “Tospearl 145-A” by | | |
| GE Toshiba Silicones | | |
| Talc | 5.5 | g |
| Pyrogenic silica, sold under the name | 1.8 | g |
| “Aerosil 200” by Degussa | | |
| Nylon powder | 2.75 | g |
| Pigments | 8 | g |
| Preservatives | 0.5 | g |

This foundation exhibited a smooth cream texture that melted when it was applied to the skin; it spread easily over the skin, without making a crunching noise, and made it possible to obtain a homogeneous make-up of the skin with a matte appearance that had a powdery finish and left the skin very soft. 12 hours after application, the make-up also exhibited good hold to sebum and to sweat.

1. An anhydrous composition for making up and/or caring for skin comprising
an oily phase;
at least one amorphous gelling polymer formed by polymerization of at least one olefin; and
at least one polyurethane powder.

2. The composition according to claim 1, wherein the at least one polymeric gelling agent is chosen from amorphous block copolymers of styrene and at least one olefin.

3. The composition according to claim 1, wherein the at least one polymeric gelling agent is formed by polymerization of ethylenic carbon monomers.

4. The composition according to claim 1, wherein the at least one polymeric gelling agent is formed by polymerization of at least one olefin chosen from ethylene, propylene, butadiene, and isoprene.

5. The composition according to claim 1, wherein the at least one polymeric gelling agent is chosen from diblock, triblock, multiblock, radial, and star copolymers.

6. The composition according to claim 5, wherein the at least one polymeric gelling agent is chosen from triblock, multiblock, radial, and star copolymers.

7. The composition according to claim 1, wherein the at least one polymeric gelling agent is chosen from optionally hydrogenated copolymers comprising styrene blocks and ethylene/C₃-C₄ alkylene blocks.

8. The composition according to claim 7, wherein the at least one polymeric gelling agent is a hydrogenated diblock copolymer chosen from styrene-ethylene/propylene and styrene-ethylene/butadiene copolymers.

9. The composition according to claim 7, wherein the at least one polymeric gelling agent is a hydrogenated triblock copolymer chosen from styrene-ethylene/propylene-styrene copolymers, styrene-ethylene/butadiene-styrene copolymers, styrene-isoprene-styrene copolymers, and styrene-butadiene-styrene copolymers.

10. The composition according to claim 1, wherein the at least one polymeric gelling agent is a blend of hydrogenated styrene-butylene/ethylene-styrene triblock copolymer and of hydrogenated styrene-propylene-styrene star polymer.

11. The composition according to claim 1, wherein the at least one polymeric gelling agent is present in an amount ranging from 0.1% to 10% by weight, relative to the total weight of the composition.

12. The composition according to claim 11, wherein the at least one polymeric gelling agent is present in an amount ranging from 1% to 3% by weight, relative to the total weight of the composition.

13. The composition according to claim 1, wherein the at least one polyurethane powder is not film-forming.

14. The composition according to claim 1, wherein the at least one polyurethane powder is chosen from powders formed of hexamethylene diisocyanate and trimethylol hexyllactone copolymer.

15. The composition according to claim 1, wherein the at least one polyurethane powder is present in an amount ranging from 0.5% to 30% by weight, relative to the total weight of the composition.

16. The composition according to claim 15, wherein the at least one polyurethane powder is present in an amount ranging from 5% to 15% by weight, relative to the total weight of the composition.

17. The composition according to claim 1, further comprising poly(methyl methacrylate) particles.

18. The composition according to claim 1, wherein the poly(methyl methacrylate) particles are present in an amount ranging from 0.5% to 30% by weight, relative to the total weight of the composition.

19. The composition according to claim 18, wherein the poly(methyl methacrylate) particles are present in an amount ranging from 5% to 15% by weight, relative to the total weight of the composition.

20. The composition according to claim 1, further comprising at least one volatile oil.

21. The composition according to claim 20, wherein the at least one volatile oil is chosen from volatile hydrocarbon oils.

22. The composition according to claim 21, wherein the volatile hydrocarbon oils are chosen from volatile hydrocarbon oils comprising from 8 to 16 carbon atoms.

23. The composition according to claim 22, wherein the volatile hydrocarbon oils are chosen from isododecane, isodecane, isohexadecane, and mixtures thereof.

24. The composition according to claim 23, wherein the volatile hydrocarbon oils are a mixture of isododecane and isohexadecane.

25. The composition according to claim 20, wherein the at least one volatile oil is chosen from volatile silicone oils.

26. The composition according to claim 25, wherein the volatile silicone oils are chosen from octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

27. The composition according to claim 20, wherein the at least one volatile oil is present in an amount ranging from 5% to 60% by weight, relative to the total weight of the composition.

28. The composition according to claim 27, wherein the at least one volatile oil is present in an amount ranging from 30% to 50% by weight, relative to the total weight of the composition.

29. The composition according to claim 1, further comprising at least one non-volatile oil.

30. The composition according to claim 29, wherein the at least one non-volatile oil is chosen from non-volatile hydrocarbon oils and non-volatile silicone oils.

31. The composition according to claim 30, wherein the at least one non-volatile oil is chosen from C₁₂-C₃₆ esters.

32. The composition according to claim 29, wherein the at least one non-volatile oil is present in an amount ranging from 0.5% to 60% by weight, relative to the total weight of the composition.

33. The composition according to claim 32, wherein the at least one non-volatile oil is present in an amount ranging from 10% to 20% by weight, relative to the total weight of the composition.

34. The composition according to claim 29, wherein the composition further comprises poly(methyl methacrylate) particles and wherein the at least one non-volatile oil is present in an amount greater than the amount of the poly(methyl methacrylate) particles.

35. The composition according to claim 34, wherein the at least one non-volatile oil and the poly(methyl methacrylate) particles are present in an amount such that the ratio of non-volatile oil to poly(methyl methacrylate) particles by weight is greater than or equal to 1:1.

36. The composition according to claim 35, wherein the at least one non-volatile oil and the poly(methyl methacrylate) particles are present in an amount such that the ratio of non-volatile oil to poly(methyl methacrylate) particles by weight ranges from 1:1 to 15:1.

37. The composition according to claim 34, wherein the at least one non-volatile oil and the poly(methyl methacrylate) particles are present in an amount such that the ratio of non-volatile oil to poly(methyl methacrylate) particles by weight is greater than or equal to 1.5:1.

38. The composition according to claim 34, wherein the at least one non-volatile oil and the poly(methyl methacrylate) particles are present in an amount such that the ratio of non-volatile oil to poly(methyl methacrylate) particles by weight ranges from 1.5:1 to 15:1.

39. The composition according to claim 1, further comprising at least one coloring material.

40. The composition according to claim 39, wherein the at least one coloring material is chosen from pigments, pearlescent agents, and fat-soluble dyes.

41. The composition according to claim 40, wherein the at least one coloring material is present in an amount ranging from 0.1% to 30% by weight, relative to the total weight of the composition.

42. The composition according to claim 41, wherein the at least one coloring material is present in an amount ranging from 5% to 15% by weight, relative to the total weight of the composition.

43. The composition according to claim 17, further comprising at least one additional filler other than the at least one polyurethane powder and the poly(methyl methacrylate) particles.

44. The composition according to claim 43, wherein the at least one additional filler is chosen from talc, mica, silica, kaolin, polyamide powders, poly-β-alanine powders, polyethylene powders, powders formed of tetrafluoroethylene polymers, lauroyllysine, starch, boron nitride, poly(vinylidene chloride)/acrylonitrile hollow microspheres, acrylic acid copolymers hollow microspheres, silicone resin powders, polyorganosiloxane elastomer particles, precipitated calcium carbonate, magnesium carbonate, basic magnesium carbonate, hydroxyapatite, hollow silica microspheres, glass and ceramic microcapsules, and metal soaps derived from organic carboxylic acids comprising from 8 to 22 carbon atoms.

45. The composition according to claim 44, wherein the at least one additional filler is chosen from silsesquioxane powders, talc, and polyamide powders.

46. The composition according to claim 43, wherein the at least one additional filler is present in an amount ranging from 0.1% to 35% by weight, relative to the total weight of the composition.

47. The composition according to claim 46, wherein the at least one additional filler is present in an amount ranging from 1% to 25% by weight, relative to the total weight of the composition

48. The composition according to claim 45, wherein the silsesquioxane powders are present in an amount ranging from 0.5% to 30% by weight, relative to the total weight of the composition.

49. The composition according to claim 48, wherein the silsesquioxane powders are present in an amount ranging from 1% to 10% by weight, relative to the total weight of the composition.

50. The composition according to claim 1, comprising a total content of pulverulent materials ranging from 20% to 50% by weight, relative to the total weight of the composition.

51. The composition according to claim 50, comprising a total content of the pulverulent materials ranging from 30% to 40% by weight, relative to the total weight of the composition.

52. The composition according to claim 1, further comprising, in the oily phase, at least one inorganic thickener.

53. The composition according to claim 52, wherein the at least one inorganic thickener is chosen from organophilic clays and pyrogenic silicas.

54. The composition according to claim 52, wherein the at least one inorganic thickener is present in an amount ranging from 0.5% to 5% by weight, relative to the total weight of the composition.

55. The composition according to claim 54, wherein the at least one inorganic thickener is present in an amount ranging from 2% to 4% by weight, relative to the total weight of the composition.

56. The composition according to claim 1, further comprising at least one additional cosmetic ingredient chosen from antioxidants, fragrances, preservatives, neutralizing agents, surfactants, sunscreen agents, vitamins, moisturizing agents, self-tanning compounds, and antiwrinkle active principles.

57. The composition according to claim 1, wherein the composition is devoid of polymer particles dispersed in the oily phase of the composition and stabilized at the surface by at least one stabilizing polymer.

58. The composition according to claim 1, wherein the composition is in a form chosen from a foundation, an eye shadow, a blusher, a concealer, a product for making up the body, a make-up base, a mattifying product for the skin, and a product for caring for the skin.

59. The composition according to claim 1, wherein the composition is provided in the form of a deformable non-fluid gel.

60. A foundation composition comprising an oily phase, at least one amorphous gelling polymer formed by polymerization of at least one olefin, and at least one polyurethane powder, wherein the foundation composition is anhydrous.

61. A cosmetic process for making up and/or for the non-therapeutic treatment of the skin, comprising applying, to the skin, at least one anhydrous composition comprising an oily phase, at least one amorphous gelling polymer formed by polymerization of at least one olefin and at least one polyurethane powder

62. A method for producing a make-up having good hold, a soft and/or comfortable feeling on made-up skin, a homogeneous appearance, and/or a powdery appearance, the method comprising applying, to the skin, at least one anhydrous composition comprising an oily phase, at least one amorphous gelling polymer formed by polymerization of at least one olefin and at least one polyurethane powder, wherein the make-up produced has at least one property chosen from good hold, soft made-up skin on application, comfort, powdery appearance, and homogeneity.
